(19) United States
(12) Patent Application Publication
(10) Pub. No.: US 2022/0395391 A1
(43) Pub. Date: Dec. 15, 2022

(54) BLADDER COLLECTION SYSTEM

(71) Applicant: TRITON SYSTEMS, INC., CHELMSFORD, MA (US)

(72) Inventors: James F. SAUNDERS, Lowell, MA (US); Tyson LAWRENCE, Highlands Ranch, CO (US); Samantha BURDETT, Lowell, MA (US); Michael J. GUARINO, Wakefield, MA (US); Cole GODZINSKI, North Reading, MA (US); Kim HALLETT, Rochester, NH (US); Connor ADAMIK, Medford, MA (US); Maciej PIETRUSINSKI, Boston, MA (US); Olivia CHONG, Somerville, MA (US)

(21) Appl. No.: 17/838,105

(22) Filed: Jun. 10, 2022

Related U.S. Application Data
(60) Provisional application No. 63/209,792, filed on Jun. 11, 2021.

(51) Int. Cl. A61F 5/451; A61F 5/44

(52) U.S. Cl. CPC A61F 5/451; A61F 5/4404; A61F 5/4401

(57) ABSTRACT

Disclosed herein is a bladder collection system. Particularly, a bladder collection system for females is disclosed. The system is designed to be flexible and useful in upright or mobile situations. The bladder relief system includes a collection interface sized and configured for seated or standing application, a sleeve, defining a collection opening, and sidewalls defining a collection cavity, an interface tubing, extending from one end of the sleeve to another, between a collection end of the interface tubing, and an exit port for connection to an exit hose; and an adsorbing material contained within the collection cavity.

### CROSS-REFERENCE TO RELATED APPLICATIONS

This application claims benefit of priority to US Provisional Patent Application Ser. No. 63/209,792 entitled BLADDER COLLECTION SYSTEM, which was filed on Jun. 11, 2021, which is hereby incorporated by reference in its entirety.

### GOVERNMENT INTEREST

This invention was made with Government support under Contract No./Job No.: N68335-19-C-0003/2589 by the U.S. Department of the Navy. The Government has certain rights in this invention.

### BACKGROUND

Pilots, particularly military pilots, will participate in tactical dehydration to avoid the urge or need to urinate inflight, increasing risks to cognitive and physical performance and mishaps. Current passive bladder relief systems require the user to remove flight gear and safety harnesses for proper collection and easier access to the device. Other passive systems such as diapers (retention garments) remain wet and uncomfortable to the user over long periods of time, frequently leading to infection. Current active systems are complex and may require inflatable seals or the initiation of urine flow in order to initiate the activation of the system. These systems are often bulky, uncomfortable, and difficult to operate. Users often lose confidence with moisture detection systems as they have delayed reaction, tend to leak, and there is no way to check the system will work properly when used.

Typical external catheters, such as that disclosed in U.S. Pat. No. 8,287,508 are designed for the medical use case for users in a supine position who usually lack the ability to move or change positions on their own. Thus, these interfaces are designed to be comfortable when used in this position. Such systems do not account for the demands of a pilot, particularly a military pilot, both in the cockpit and out of it.

Accordingly, there is a need for more and better bladder relief systems particularly for use by pilots, particularly military pilots.

### SUMMARY

Disclosed herein is an external catheter system having a collection interface designed and configured to account for the special needs required for a seated and/or mobile user, such as, but not limited to pilots.

Some embodiments provide a bladder relief system comprising a collection interface sized and configured for seated or standing application, the collection interface further comprising a sleeve, defining a collection opening, and sidewalls defining a collection cavity, an interface tubing, extending from one end of the sleeve to another, between a collection end of the interface tubing, and an exit port for connection to an exit hose; an adsorbing material contained within the collection cavity; an exit hose, fluidly connected to said interface tubing and a collection receptacle; and a pump, for facilitating flow of fluid from the collection cavity through the interface tubing, exit hose, and into the collection receptacle.

In some embodiments, the collection interface, and its subcomponents, the exit hose, and collection receptacle are disposable.

In some embodiments, the collection device is provided with a quick disconnect system to be readily disconnectable from the remainder of the system without leakage.

### BRIEF DESCRIPTION OF THE DRAWINGS

Aspects, features, benefits and advantages of the embodiments described herein will be apparent with regard to the following description, appended claims, and accompanying drawings where: The present disclosure is described with reference to the following figures, which are presented for the purpose of illustration only and are not intended to be limiting.

In the drawings:

FIG. 1 is a schematic drawing of the bladder relief system in accordance with some embodiments disclosed herein.

FIG. 2 is a drawing of various components of the bladder relief system in accordance with some embodiments disclosed herein.

FIG. 3 is a schematic drawing of a portion of the bladder relief system in accordance with some embodiments disclosed herein.

FIG. 4 are alternative embodiments of the collection interface of the bladder relief system in accordance with some embodiments disclosed herein.

FIG. 5 is a photo of the collection interface with exit hose of a bladder relief system in accordance with some embodiments disclosed herein.

FIG. 6 is a photo of the collection interface of the bladder relief system in accordance with some embodiments disclosed herein.

FIG. 7 is series of photos of the collection end and notch of the interface tubing of the bladder relief system in accordance with some embodiments disclosed herein.

FIG. 8 is a schematic drawing of a collection receptacle of the bladder relief system in accordance with some embodiments disclosed herein.

FIG. 9 is a photo of a peristaltic pump in accordance with some embodiments disclosed herein.

FIGS. 10 and 11 are, each, a schematic drawing a peristaltic pump in accordance with some embodiments disclosed herein.

FIG. 12 depicts a variety of tubing management solutions in accordance with some embodiments.

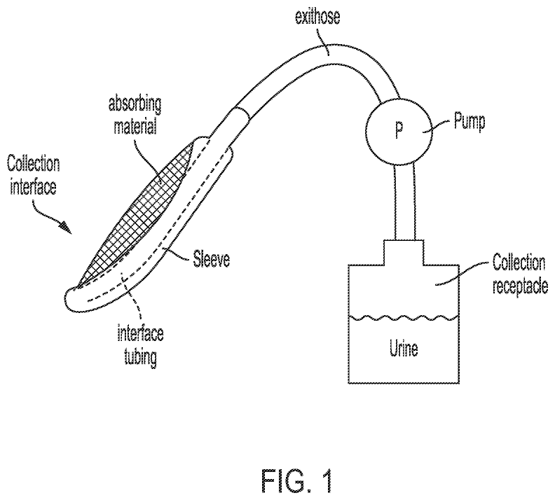

### DETAILED DESCRIPTION

The system disclosed herein provides portable bladder relief to pilots of all sexes while in flight and maintaining their situational awareness and performance in-flight. While using an external collection interface with a portable pump system, pilots will be able to relieve themselves while seated in an aircraft. The perceived need for tactical dehydration will be reduced or eliminated.

It will be appreciated that for clarity, the following discussion will describe various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person in the art will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

Various terms are used herein consistent with their common meanings in the art. The following terms are defined below for clarity.

It must also be noted that as used herein and in the appended claims, the singular forms “a”, “an”, and “the” include plural reference unless the context clearly dictates otherwise. Thus, for example, reference to “a device” is a reference to “one or more devices” and equivalents thereof known to those skilled in the art, and so forth.

As used herein, the term “about” means plus or minus 10% of the numerical value of the number with which it is being used. Therefore, “about 50” means in the range of 45-55.

It will be appreciated that while a particular sequence of steps is shown and described herein for purposes of explanation, the sequence may be varied in certain respects, or the steps may be combined, while still obtaining the desired configuration. Additionally, modifications to the disclosed embodiment and the invention as claimed are possible and within the scope of this disclosed invention.

The bladder relief system disclosed herein includes a collection interface, a collection receptacle 200, a control unit, and a pump system. Additional or alternative elements or modules may be employed.

Collection Interface

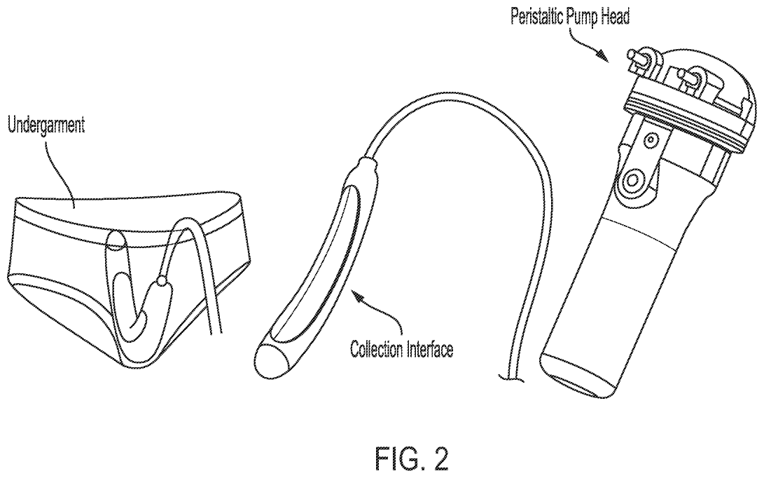

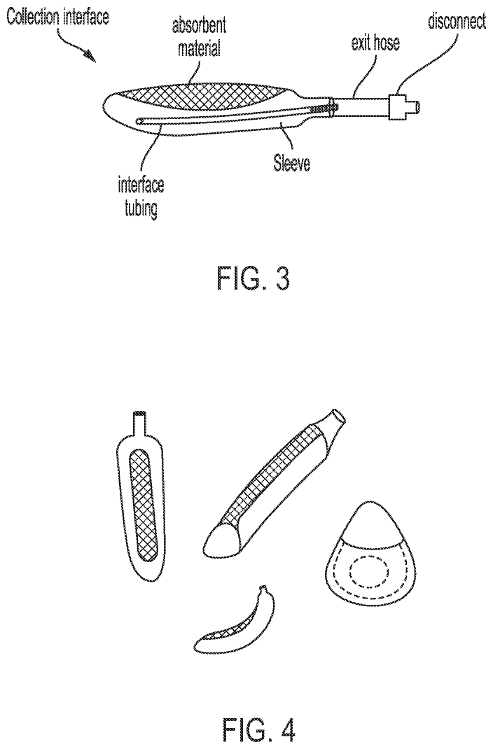

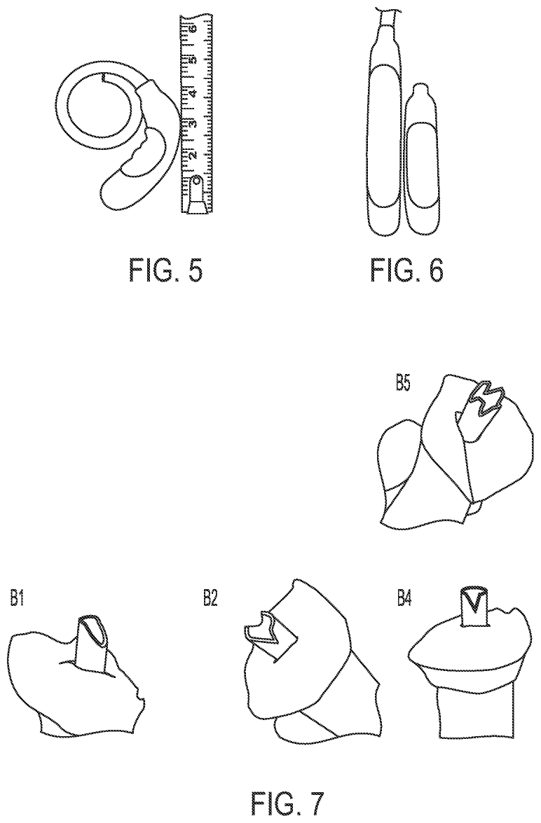

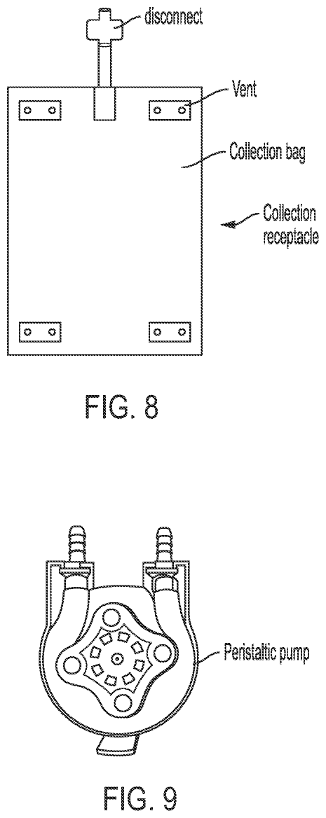

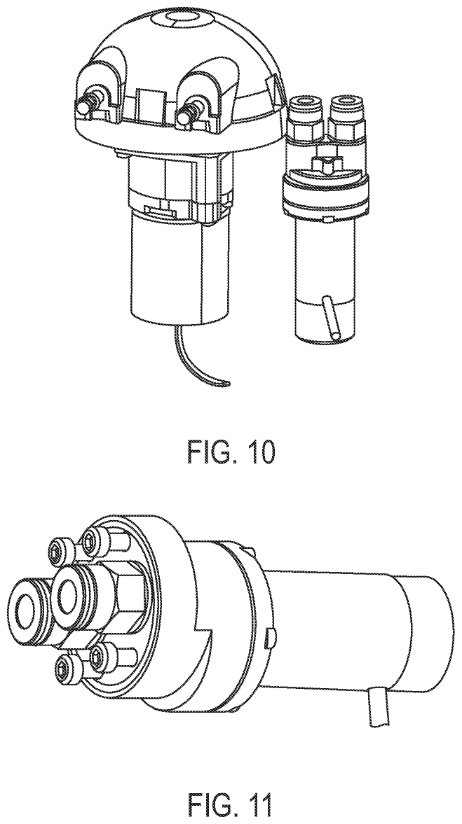

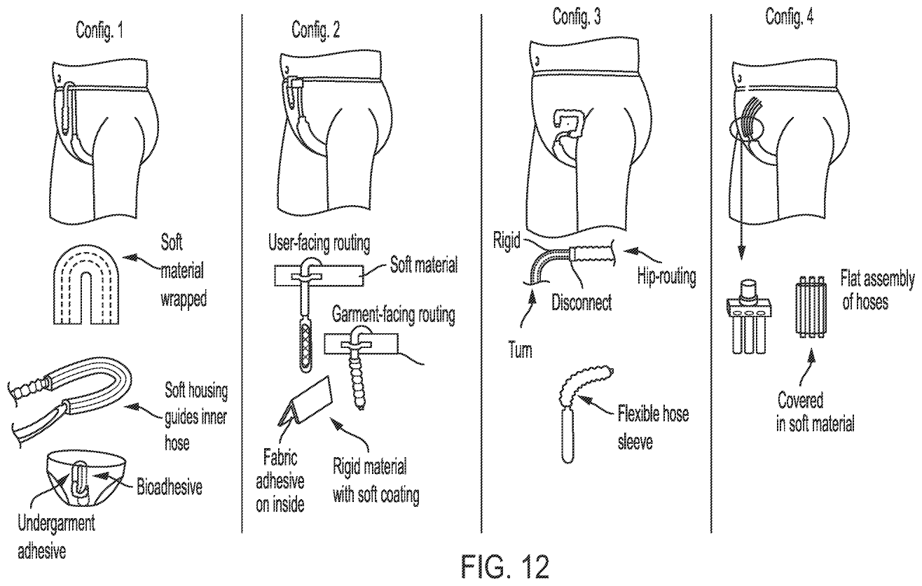

Existing external catheters do not conform comfortably to the female anatomy in a seated position. By “female anatomy” the description herein refers to those users having a vulva regardless of assigned sex at birth. The interface disclosed herein is relatively short for comfort and fit. It is designed to be a suitable length for seated users. Various widths or diameters are possible, as are different filter/wicking materials. The system may also be used with a modified undergarment (e.g. see FIGS. 2 and 12) for tube routing (an optional retention garment).

The device is adapted with reinforcement to conform to the anatomy without shifting or twisting during movement and/or use. The interface is shaped to be worn and maintain position during activity (standing, walking, squatting, etc.). Additional material, such as but not limited to moisture wicking fabrics, in the kit can be added to the interface to increase comfort and confidence in the system. An optional touch indicator such as but not limited to a raised element allows users to locate the orientation of the interface an align properly with the body.

External catheters have existed for many years, but do not work well in the sitting or standing position due to their design. Further, they are not well-adapted to active users such as pilots who need to climb in and out of their cockpits, run on the tarmac or in the field, etc. Existing catheters are designed essentially for bed-ridden medical patients. This disclosure identifies several novel improvements, including but not limited to combinations of size and shape, that have not been used before to achieve superior performance to existing products.

Some embodiments provide a more secure the interface for users who are mobile. A specialized garment could add security, and also have some element of absorption to it. Additional ideas for keeping the interface secure include adding a texture (e.g., raised bumps) to the underwear-side of the interface, or an adhesive patch that would stick to underwear.

The overall form factor or shape of the collection interface 100 may take one of several shapes including tear drop, tube, curved tube, or a more complex shape. The overall interface (minus an exit hose) is from about 2 inches to about 7 inches in length, which comfortably accommodates users with vulvas and across a variety of anatomical sizes. (see e.g. FIG. 5) Specifically, this anthropometric measurement is based on the distance between the urethra and perineum. Three distinct interface lengths have been identified to increase comfort for bodies of different sizes, and when worn while seated and wearing civilian street clothing or military clothing (e.g., jeans, military gear, PPE, etc). This is in contrast to an existing “one-size-fits-all” product which is comfortable primarily when used with loose hospital gowns in the supine position but creates discomfort in the above use case.

In a tubular or tube-like form, the collection interface 100 is from about 0.5inch to about 1 inch in diameter, with optional configurations including, but not limited to those that taper at the front and on the bottom to create a teardrop shape. See FIG. 4 for exemplary shapes. The taper provides increased comfort for users with smaller labia and for all wearers when used in a seated posture. The collection interface 100 may be other shapes including but not limited to hourglass, teardrop, tubular, frusto-conical, etc.

Additionally, a curve may be added to the overall collection interface 100 to increase comfort and fit when used in a seated posture.

The collection interface 100 comprises a sleeve 102, having an opening for receiving urine and defining an inner chamber for collecting urine. The sleeve 102 maybe made of any liquid (e.g. urine) impervious material, such as rubber, silicone, a breathable sheath (e.g. Goretex), etc. The idea is to provide a chamber to prevent urine from reaching the pilots clothes or skin before reaching the exit tube. Within the chamber is absorption and or wicking material, as well as inner tubing for directing the urine out of the chamber and away from the user. The sleeve 102 of the collection interface 100, as noted above, may include an embossed rim for tactile indication of wick orientation for users with impaired vision. The sleeve 102 maybe be any suitable water tight material, such as silicone. In some embodiments, the sleeve 102 will be flexible to accommodate the users natural shape. In other embodiments, the sleeve 102 may have one or more fixed dimensions and/or shape so as to maintain its shape.

In some instances, a tactile indicator is designed, configured and oriented such that it can be felt against skin to identify proper location. Alternatively, a tactile indicator designed, configured and oriented such that it can be felt through clothing from the outside as a landmark. The tactile indicator can be a raised bump or series of bumps, on the exit hose 108 end of the interface, that allow the user to determine proper orientation without visual inspection. Other configurations could be workable.

Absorption Material

An absorbing material 104 is placed within the chamber of the sleeve 102 to minimize leakage. The absorbing material 104 may be any suitable absorbent material, such as but not limited to cotton, and may further include additional material, layers, treatments, such as hydrophobic or hydrophilic treatments, nanomaterial composites, etc. to achieve additional desired properties. The absorbing material 104 is substantially completely contained within the sleeve 102. The absorbing material 104 is about 3 to about 5 inches in length, consistent with the overall size of the collection interface 100, which will comfortably accommodate users with vulvas and across a variety of anatomical sizes. Three distinct sizes have been identified to increase comfort for bodies of different sizes particularly for active users who don the interface underneath civilian street clothing or military clothing (e.g., jeans, military gear, PPE, etc) and then need to walk, run, squat, etc. Improved materials which are smoother than cotton are helpful to reduce shear and friction to increase comfort for active users by mitigating chafing. Although retentive materials, such as those used in diapers maybe used, they do not facilitate removal of urine from the collection device and are bulky and uncomfortable when wet.

Tubing

The size, shape, stiffness of the tubing may vary, taking into account the desired properties such as but not limited to flow-rate, internal diameter, external diameter, curvature required, resiliency, etc.

Softer materials such as but not limited to silicone multilumen tubing are soft while resisting kinking/crushing that could block flow are suitable for some applications. Other kink resistance techniques and materials may be employed. For example, a composite structure to limit minimum bend radius while allowing use of a soft tube material maybe employed. Other materials contemplated include but are not limited to inflatable tubing, flat tubing (single or multiple), corrugated tubing, spiral wire tubing, variable diameter tubing, coiled tubing, etc. It is contemplated that different types of tubing may be used in different portions.

Tubing may be provided with a cover or sheath to add protection, comfort, or other desirable property.

Tubing design can be chosen to favor certain flow regimes, e.g. bubbly, slug, annular, stratified—e.g. drip chamber, venturi, etc.

Interface tubing

Within the sleeve 102, an length of interface tubing 106 is provided to allow urine to flow from the sleeve cavity and absorbing material 104 through the interface tubing 106e and eventually to a collection receptacle 200. The interface tubing 106 is notched at one end to prevent leaks and occlusion and to allow urine to enter the interface tubing 106 for removal. The tube material is chosen for comfort and fit. Different notch 107 patterns may be used to mitigate occlusion while maintaining a high flow rate. Notch 107 chosen (‘B4’ below) provides optimal occlusion mitigation and maintains the highest flow rate.

The interface tubing 106 is from about 1/8 to about 1/4 inch ID, and between 1/4—3/8 inch OD. Smaller diameters increase comfort when worn in a seated position, but may restrict flow. The addition of a notch 107 on the interface tubing 106 mitigates an issue with suction at the back of the sleeve when it is used in a seated posture.

Exit Hose

An exit hose 108 can be connected to the interface tubing 106 or may be an extension thereof. The exit hose 108 extends from the collection interface 100 to the collection receptacle 200 fluidly communicating urine from the former to the latter. The exit hose 108 may be any length appropriate to connect the collection interface 100 and collection receptacle 200 while accommodating a pump 200 therebetween. In some embodiments, the exit hose 108 is from about 3 to about 18 inches in length. A 3″ exit hose 108 provides easy access at the bottom of the flight suit. An 18″ length provides easy access at the top of the flight suit. Additional lengths of hosing and appropriate connectors 400 may be employed to add flexibility in use and placement of other components of the system, or to facilitate detachment without leaving an uncomfortable length of exit hose 108.

Connectors

A pair of valved, no-drip, mated quick-disconnect connectors 400 may be added to the interface tubing 106, exit hose 108, or additional lengths of tubing to facilitate connection but also to maintain a seal when disconnected from one another. This minimizes or prevents urine from spilling after use. Such connectors 400 also support single-handed use for military pilots who must maintain a hand on their controls at all times.

In some embodiments, the connectors 400 are designed and configured for easily and reliably getting a hose in and out of pump 200 (e.g. a peristaltic pump) in the right direction (e.g. mating piece on tube). This may be accomplished in a number of ways, including but not limited to allowing providing specific mated connections to ensure tubing is connected in the correct location and in the correct direction.

Collection Receptacle

Although any type of collection receptacle 200 may be used, collection bags are contemplated since they are light weight, disposable, and easily stored. In some embodiments, a collection receptacle 200 is a bag having about 1 Liter capacity. This is enough for holding either one “very full bladder” void, or up to three “starting to feel the urge to go” voids depending on the individual user. Other sizes maybe be used. As with the interface tubing 106 and exit hose 108 described above, the collection receptacle 200 may be provided with a mated valved, no-drip, quick-disconnect connector to allow for quick connection to and disconnection from the exit hose 108. The quick-disconnect enables users to easily swap out collection bags. It also supports single-handed use for military pilots who must always maintain a hand on their controls. Additionally, a duck bill or other type of check valve in the collection bag port may be employed to prevent back flow of urine.

The collection bag may be provided with an exit port at the bottom of the collection bag (not shown in drawing) to allow for multiple uses of the bag if needed. Additionally, military users on aircraft carriers can adhere to the protocol of emptying the collection bag before disposing of it using the ship's waste restrictions. There may be a user-friendly, easy-to-use sliding motion that opens and closes the port. Other embodiments will employ disposable collection receptacles.

In some embodiments, a gelling component may be used within the collection receptacle to prevent leaks of urine post urination. This is ideal for transport where users may need to store and carry their collection bag to nearest disposal. It will also minimize “liquid sloshing around” in the cockpit which is disfavored by pilots.

The collection receptacle may also be provide with one or more air vents to prevent the collection receptacle from inflating if the pump 200 pulls air during urination. This allows the collection bag to be held or laid in any orientation while system is in use. In some embodiments, from 1 to eight vents may be used. To minimize liquid leakage, the vents can be valved. Helpful for users with visual impairment, and/or military pilots who need to maintain visual focus on their control s/surroundings.

Tubing management

Not surprisingly, with an upright, mobile user, tubing management is more important than with an immobile bed-ridden user. FIG. 12 shows a few potential routing paths and techniques for routing the exit tubing.

In some embodiments, a relatively short exit tube will be connected to the collection device, and end with a connector close to or integrated with the users outer garment or flight suit. That connector is used to connect to a length of tubing leading eventually to the pump 200. That connector is preferably a self-sealing connector to prevent any liquid from entering or exiting the tubing. Prevention of liquid entering the exposed end of the exit tube is not desirable for many reasons, the least of which is it would allow water to enter the device should the user find themselves is water, such as in the case of an emergency water landing or other situation.

In the embodiments shown, a length of exit tubing exits an undergarment either at the waist band or at the leg. The “Config. 4” shown employs a series of flat tubing which may aid in comfort by reducing bulk. It may also help in reducing movement of the tubing. Other designs shown rely on clips or other features to secure the tubing.

Control Units and Pumps

The systems contemplated herein may use any of a variety of controls or combination thereof to activate or deactivate the system. The pump 200 can be run continuously to evacuate liquid immediately, where pump speed could increase upon triggering a moisture sensor, the pump could be activated by a moisture sensor, deactivation may be by timer, activation/deactivation may be by user input (e.g. a button or switch, leg activated sensors, etc.) The device may be provided with a visual indicator (e.g. a light) to let the user know it is running. Sensors and indicators may also be provided to alert the user to a blockage.

Because the devices contemplated herein are meant to be portable, they should be relatively small and light weight. With respect to size, the pump should be relatively small and compact for ease of storage, portability, with a minimal footprint so as to take up minimal room in an already crowded cockpit. Similarly, weight should be limited to accommodate portability, and flight considerations. Runtime should be sufficient to account for maximum maneuver time, this may depend on the situation, aircraft (or other vehicle), ability to re-fuel mid-maneuver, etc. The flow rate should be sufficient to withdraw urine from the user to limit pooling and minimizing wetting of the user's skin or clothing, exemplary flow rate is 25mL/s. All properties of the pump and other system elements, separately and together, should take into account portability, weight, size, and performance characteristics, particularly those required in military applications.

Any suitable pump system may be used. Vacuum-based pump systems require AC power and are typically not portable. Noise is also a concern given the intended use in a small cockpit in relatively close proximity to the user. Non-vacuum systems, such as peristaltic pumps or gear pumps, are therefore preferred. Other types of pumps, such as but not limited to diaphragm, flexible vane, centrifugal, etc. may be used. With a peristaltic pump the portions that contact the urine are mostly disposable components which mitigates the need to clean the pump. Fluid does not come in contact with the pump itself, making it a mess-free option. A gear pump is quiet, small, light-weight, and self-priming. Both pumps are portable, have a good battery life, and they meet the SWaP constraints for the user.

Motor control can be adjusted to allow for intermittent or turbulent flow. In some embodiments, the pump is optimized for maintaining constant flow despite variations in pressure head (resistance to flow). In some instances, such pumps have difficulty handling intermittent flow (e.g. presence of air in the line to overcome this limitation, the constant flow feature is disabled (so make it ‘dumber’) and a limit on motor speed added so that the pump motor does not rev way up when an air pocket hits it. This will help keep the system more discreet (no wild noise fluctuations) and less startling to the user. This is a feature—pump motor speed limit to maintain smooth and quiet operation despite air in the supply line—is novel.

In some embodiments, the pump may be operated in reverse to allow for an influx of air to facilitate drying. In some embodiments, particularly those for use with sealed suits, the system is provided with an air intake to allow for pressures to equalize in the suit as material is removed.

Peristaltic pumps work by repeatedly squeezing fluid through a tube. They have no complex parts, can push or pull fluid up large distances, and are very robust to tubing kinks. However, in this application, their distinct advantage is hygiene. In most pump types, fluid (in this case urine) flows through the internal structure of the pump during operation. The complicated surfaces inside the pump would retain fluid and are difficult to clean and must be flushed regularly with fresh water. Peristaltic pumps, on the other hand, push fluid through a flexible tube. No part of the pump ever comes into contact with the fluid, and the tube can be disposed of after each use. Tubing selection is important, but no particular special materials are needed for peristaltic function and thus this section of tubing does not impose excess cost to be disposable.

This control unit uses a peristaltic pump. The gearing of the peristaltic pump is adapted to increase flow rate of at least 25 ml/sec. This flow rate accommodates average human urine flow rates, so that urine may be removed almost as quickly as it is released.

The pump will be required to move urine from the base of the seat to the collection receptacle, wherever it may be. In most cases, it is expected that pilots will place the collection bag and pump in their lap (if convenient), beside them, hanging from their MOLLE vest, or behind them in their helmet bag.

A gear pump also meets the needs in terms of size and weight and can be modified to reach a minimum flow rate of 25 ml/sec. The pump was also intended for panel mounting and did not have adequate mounting points or waterproofing options for a handheld design. A standard gear pump was modified to match the flow and pressure specs, as well as in-flight mounting requirements. The custom pump can be mounted to the enclosure with four cap screws, and an O-ring generates a tight seal at the interface.

It provides a small overall form factor that is good for portability. It fits easily in G-suit pocket of military fighter pilots. It is also lightweight for increased portability and not a burden to carry. Night Vision Goggle-compatible Light-Emitting Diodes (LEDs) may be used to indicate battery life and pump operation. These provide compatibility for nighttime military missions. Use of O-rings and gaskets makes this pump water resistant. A clip on the back of the control unit allows for control unit to be stowed in pocket securely or clipped on clothing/gear while in use (i.e. MOLLE vest of military users).

Optional rim embossed buttons are easy to use with gloves (military use case), or for users with impaired hand mobility.

Current bladder relief approaches use moisture detection to auto-start the pump. This is not desired by users because they must start urinating enough to turn the pump on, but then stop and wait for it to turn on before they can finish urinating. It's hard to stop urinating after you start urinating. Pumps contemplated herein do not use moisture detection, thus increasing confidence in use. This is also useful for users that do not need a constant vacuum (hospital use case), or suffer from incontinence. In some embodiments, the user may turn the pump on and off at times and intervals of their choosing.

The pump will be required to move urine from the base of the ejection seat to the storage bladder, wherever it may be. In most cases, it is expected that pilots will place the collection bag and pump in their lap (if convenient), beside them, hanging from their MOLLE vest, or behind them in their helmet bag.

Many current active extraction systems require cleaning. The system described herein makes use of many disposable parts. In some embodiments, the extraction pump does not come in contact with the urine, and therefore can be re-used without the need for cleaning, and without contaminating a cockpit or suit. The system contains mechanisms to prevent occlusion of the device tube and aid in maintaining a constant flow.

What is claimed is:

1. A bladder relief system comprising:
a collection interface sized and configured for seated or standing application, the collection interface further comprising:
a sleeve, defining a collection opening, and sidewalls defining a collection cavity,
an interface tubing, extending from one end of the sleeve to another, between a collection end of the interface tubing, and an exit port for connection to an exit hose 108;
an adsorbing material contained within the collection cavity;
an exit hose, fluidly connected to said interface tubing and a collection receptacle; and a pump, for facilitating flow of fluid from the collection cavity through the interface tubing, exit hose, and into the collection receptacle.

2. The bladder relief system, wherein the collection interface, and its subcomponents, the exit hose, and collection receptacle are disposable.

3. The bladder relief system wherein the collection device is provided with a quick disconnect system to be readily disconnectable from the remainder of the system without leakage.
